(19) United States
(12) Patent Application Publication
(10) Pub. No.: US 2021/0055291 A1
(43) Pub. Date: Feb. 25, 2021

(54) DETECTION METHOD OF ANTIGEN-ANTIBODY REACTION

(71) Applicant: Tunghai University, Taichung (TW)

(72) Inventor: Po-Ling Chang, New Taipei City (TW)

(21) Appl. No.: 16/547,543

(22) Filed: Aug. 21, 2019

(51) Int. Cl. G01N 33/543; G01N 21/75; G01N 21/51

(52) U.S. Cl. CPC G01N 33/54346; G01N 2021/7773; G01N 21/51; G01N 21/75

(57) ABSTRACT

A detection method of antigen-antibody reaction, including: Providing an antibody solution, including an antibody and a type of metal nanoparticle, the type of metal nanoparticle forms bond with the antibody. Adding an antigen to the antibody solution to form a mixed solution. Providing a light beam to the mixed solution, wherein part of the light beam is scattered by the type of metal nanoparticle to form a scattered light beam. Detecting the scattered light beam to determine whether an antigen-antibody reaction occurs.

### FIELD OF THE INVENTION

The present invention relates to a detection method, and more particularly to a detection method of antigen-antibody reaction.

### BACKGROUND OF THE INVENTION

ABO blood group and Rh blood group are the most common type of blood typing for blood transfusion. The conventional blood typing is performed by adding the antibodies and waiting for the agglutination of red blood cells (erythrocytes) to be visible to the naked eyes. However, the degree which the red blood cells are agglutinated to be visible to the naked eye requires a certain reaction time. When an emergency situation occurs, such as traffic accident, massive bleeding, etc., the time to wait for the agglutination reaction is easy to miss the timing of rescue.

Recently, several improved methods had been developed for the agglutination of red blood cells. For example, test paper of blood group which is more convenient in use, or automated blood group analytical device that may save labor costs. However, the above methods are still based on red blood cell agglutination. Although it may save operating time or labor costs, it may not reduce the waiting for agglutination reaction to be visible to the naked eye, which does not really help the dilemma of detection timeliness encountered in an emergency.

### SUMMARY OF THE INVENTION

The invention provides a detection method of antigen-antibody reaction, which may reduce the detection time.

A detection method of antigen-antibody reaction provided in an embodiment of the invention includes: providing an antibody solution, including an antibody and a type of metal nanoparticle, the type of metal nanoparticle forms bond with the antibody. Adding an antigen to the antibody solution to form a mixed solution. Providing a light beam to the mixed solution, wherein part of the light beam is scattered by the type of metal nanoparticle to form a scattered light beam. Detecting the scattered light beam to determine whether an antigen-antibody reaction occurs.

In one embodiment of the invention, the type of metal nanoparticle includes gold nanoparticle.

In one embodiment of the invention, the antigen includes at least one of A type antigen, B type antigen, D antigen, C antigen, E antigen, c antigen, and e antigen on red blood cells.

In one embodiment of the invention, the antibody includes a specific antibody.

In one embodiment of the invention, the method to determine whether an antigen-antibody reaction occurs includes detecting whether an intensity of the scattered beam exceeds a threshold, and it is determined that an antigen-antibody reaction occurs if the intensity exceeds the threshold.

In one embodiment of the invention, the method to detect the scattered light beam includes using a detection device including a light source and an objective type dark field microscope.

In one embodiment of the invention, the objective type dark field microscope includes a slide, a microscope objective, a mask and a first light sensing element (digital camera). The slide is adapted to carry the mixed solution. The microscope objective is disposed on a side of the slide away from the mixed solution, the light beam passes through the microscope objective by a specific angle and is irradiated to the mixed solution. The mask is disposed on a transmission path of the scattered light beam and is adapted to allow the scattered light beam to pass and block a passage of the remaining light beams. The first light sensing element is disposed on the transmission path of the scattered light beam and is adapted to receive the scattered light beam.

In one embodiment of the invention, the method to detect the scattered light beam includes using a detection device including a light source and a capillary column detection device.

In one embodiment of the invention, the capillary column detection device includes a capillary column, a microscope objective, a mask and a second light sensing element. The mixed solution is placed in the capillary column. The microscope objective is disposed adjacent to the capillary column, the light beam passes through the microscope objective by a specific angle and is irradiated to the mixed solution. The mask is disposed on a transmission path of the scattered light beam and is adapted to allow the scattered light beam to pass and block a passage of the remaining light beams. The second light sensing element is disposed on the transmission path of the scattered light beam and is adapted to receive the scattered light beam.

In one embodiment of the invention, the second light sensing element includes a photomultiplier.

In the detection method of antigen-antibody reaction of the embodiment of the invention, there is a bond between the antibody and the type of metal nanoparticle. When the antibody reacts with the antigen to form an antigen-antibody, since the type of metal nanoparticle itself may scatter the light beam and a large amount of the type of metal nanoparticle causes the scattered light beam to be greatly increased, by detecting the enhanced signal of the scattered light beam, it is possible to quickly determine whether an antigen-antibody reaction occurs, and there is no need to wait for the antigen-antibody reaction to be visible to the naked eyes. In addition, the antigen detecting process of the invention does not require any washing step, so that the presence of the antigen may be detected in a minimum of 5 seconds, and the time required to detect the antigen-antibody reaction may be greatly reduced.

Other objectives, features and advantages of The invention will be further understood from the further technological features disclosed by the embodiments of The invention wherein there are shown and described preferred embodiments of this invention, simply by way of illustration of modes best suited to carry out the invention.

### BRIEF DESCRIPTION OF THE DRAWINGS

The present invention will become more readily apparent to those ordinarily skilled in the art after reviewing the following detailed description and accompanying drawings, in which:

FIG. 1 is a schematic flow diagram of a detection method of antigen-antibody reaction of one embodiment of the invention;

FIG. 2A is an experimental picture of a detection result of a red blood cell observed by a scanning electron microscope;

FIG. 2B is an experimental picture of a detection result of a red blood cell and gold nanoparticle observed by a scanning electron microscope;

FIG. 2C is an experimental picture of a detection result of a A type red blood cell and gold nanoparticle bonding with anti-B antibody observed by a scanning electron microscope;

FIG. 2D is an experimental picture of a detection result of a A type red blood cell and gold nanoparticle bonding with anti-A antibody observed by a scanning electron microscope;

FIG. 3 is a schematic diagram of a detection device of one embodiment of the invention;

FIG. 4A to FIG. 4D are experimental pictures of detection results of ABO blood group system using a detection device of FIG. 3;

FIG. 5 is a schematic diagram of a scattering intensity result of FIG. 4;

FIG. 6A to FIG. 6E are experimental pictures of detection results of Rh blood group system using a detection device of FIG. 3;

FIG. 7 is a schematic diagram of a detection device of another embodiment of the invention; and

FIG. 8 is a schematic diagram of a detection result using the detection device of FIG. 7.

### DETAILED DESCRIPTION OF PREFERRED EMBODIMENTS

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

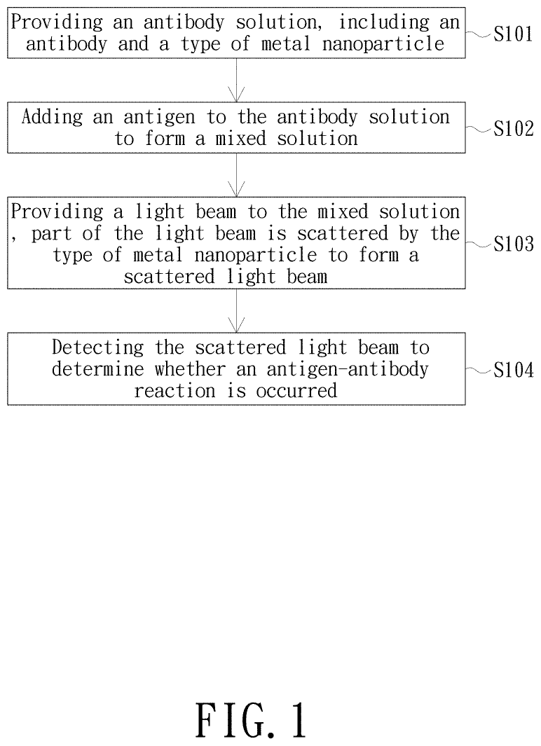

FIG. 1 is a schematic flow diagram of a detection method of antigen-antibody reaction of one embodiment of the invention. Referring to FIG. 1, a detection method of antigen-antibody reaction of the embodiment includes the following steps. Step S101: providing an antibody solution, including an antibody and a type of metal nanoparticle, the type of metal nanoparticle forms bond with the antibody. Specifically, the type of metal nanoparticle is, for example, a metal having a function of scattering light, and in the embodiment, for example, a gold nanoparticle is used. Next, step S102: adding an antigen to the antibody solution to form a mixed solution.

As used herein, the terms “metal nanoparticle”, “gold nanoparticle”, “antigen”, “antibody” and the like described throughout the present invention shall be regarded as a general term for these substances, not the actual quantity thereof. For example, the quantity of metal nanoparticle and antibody in the antibody solution is plural, and the quantity of antigen is also plural.

In the embodiment, for example, an antigen and an antibody contained in human blood are used as a detecting material. Specifically, the antigen includes at least one of A type antigen, B type antigen, D antigen, C antigen, E antigen, c antigen, and e antigen on human red blood cells, that is, the antigen of the conventional ABO blood type system and the conventional Rh blood type system; the antibody includes a specific antibody corresponding to the above antigen (that is, anti-A antibody, anti-B antibody, anti-D antibody, anti-C antibody, anti-E antibody, anti-c antibody, and anti-e antibody), but is not limited thereto. The antigen may be any organic matter carried by a living body or other substance which will react with the antibody, such as bacteria, mold, virus, medicine, pollen and the like. The antibody may also be a non-specific antibody depending on the detecting requirements. Specific embodiments of the detection method of antigen-antibody reaction will be further described below, but the specific structure of the detection method of antigen-antibody reaction of the invention is not limited to the embodiments listed below.

Regarding the preparation of the antibody solution (S101), taking the embodiment as an example, the preparation process is as follows: 25 μL 3-Mercaptopropionic acid (MPA) was added into 250 mL of synthesized gold nanoparticle (AuNP) to bring a final concentration of 10 mM and stirred it for overnight. The size of the gold nanoparticle used in the embodiment is 32 nm. For the preparation of the gold nanoparticle solution, refer to the article of Huang et al. Aptamer-modified gold nanoparticles for targeting breast cancer cells through light scattering. J. Nanopart. Res. 2009, 11 (4), 775, which will not be described in detail herein. The 100 μL MPA capped AuNP was transferred to an ethanol-prewashed PCR tube and centrifugated at 400×g for 30 minutes. After remove the suspension, the pellet of MPA-AuNP was then resuspended by 70 μL phosphate buffer (1 mM, pH 7.0) for antibody conjugation. 2 ml of blood group specific antibodies were washed three times by phosphate buffer saline (PBS) via 100 k cut-off centrifugal filter at 4000×g for 15 minutes, in order to remove the bovine serum albumin (BSA), pigment and sodium azide from antibody stock solution. The antibodies were then resuspended by 1 mM phosphate buffer (pH 7.0) and stored in a 1.5 mL eppendorf tube at 4° C. no longer than two days. The concentration of washed antibodies were about 20 μg/μL. For blood group specific antibodies functionalized gold nanoparticle (BG-AuNP) preparation, 10 μL antibodies was added to 70 μL MPA-AuNP solution. After gentle vortex and stand for 10 minutes, 10 μL 1-(3-Dimethylaminopropyl)-3-ethylcarbodiimide (EDC, 100 mM) and 10 μL sulfo-N-hydroxysulfosuccinimide (sulfo-NHS, 10 mM) were added into this mixture to bring a final volume of 100 μL and vortexed for 1 hour for antibody coupling on AuNP. The BG-AuNP was then washed four times by PBS contains 5% BSA at 600×g for 30 minutes. The BG-AuNP was stored at 4° C. and stable at least for one month. The completed BG-AuNP is the antibody solution of the embodiment.

The blood group specific antibodies (anti-A antibody, anti-B antibody, anti-D antibody, anti-C antibody, anti-E antibody, anti-c antibody, and anti-e antibody) were commercially available from Immucor Gamma (Norcross, USA). The red blood cells for ABO typing and for Rh typing were commercially available from Formosa Biomedical Technology Corp. (Taipei, Taiwan).

After the antibody solution which the antibody bonds to gold nanoparticle is configured, 1 μL of red blood cells may be directly added into 9 μL of the antibody solution to form a mixed solution (S102). Next, step S103: providing a light beam to the mixed solution, wherein part of the light beam is scattered by the type of metal nanoparticle to form a scattered light beam. According to Mie scattering theory, a scattered light beam is produced when a particle size is much smaller than a wavelength of incident light. On the other hand, Mie scattering occurs when the particle size increases to be close to or greater than the wavelength of incident light. Therefore, proceeding to step S104: detecting the scattered light beam to determine whether an antigen-antibody reaction occurs.

The method to determine whether an antigen-antibody reaction occurs includes detecting whether an intensity of the scattered beam exceeds a threshold, and it is determined that an antigen-antibody reaction occurs if the intensity exceeds the threshold. The principle of determining the antigen-antibody reaction is described in detail below.

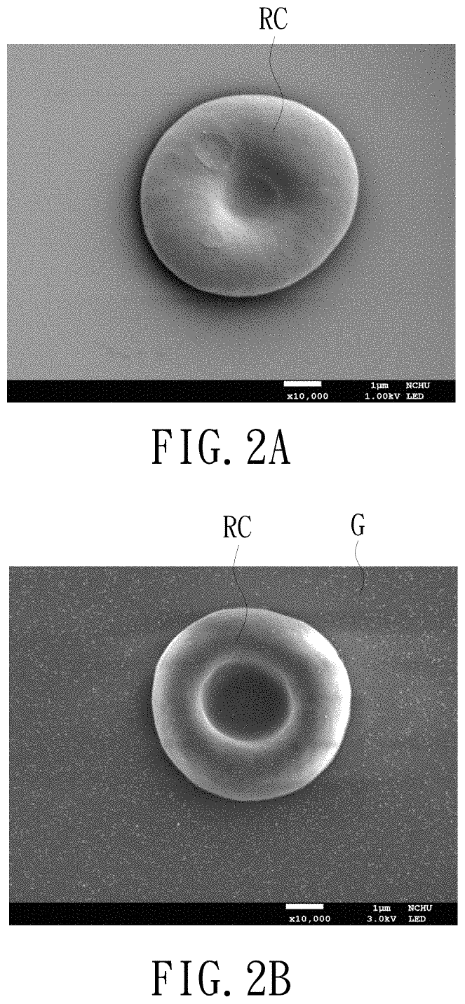

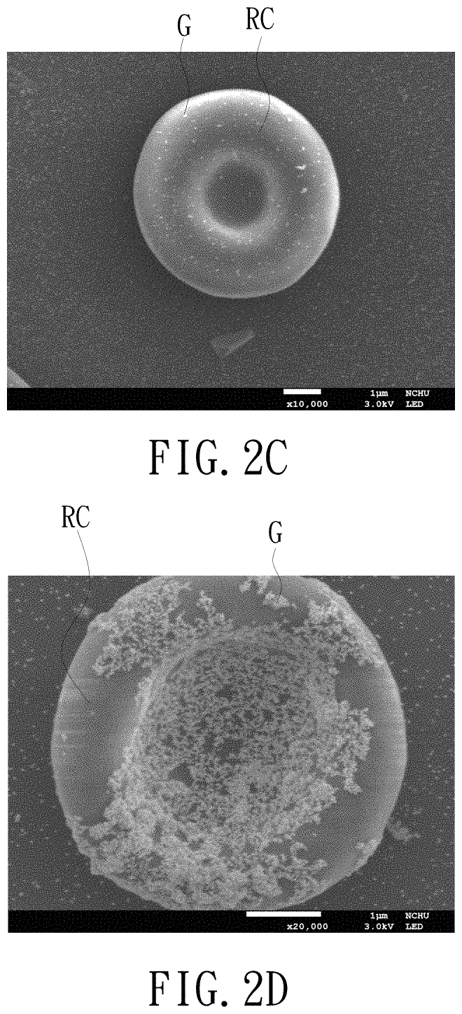

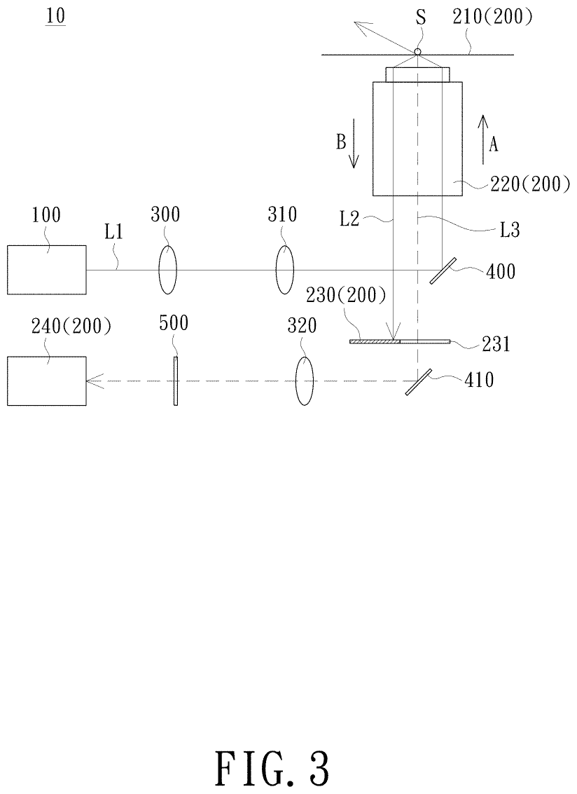

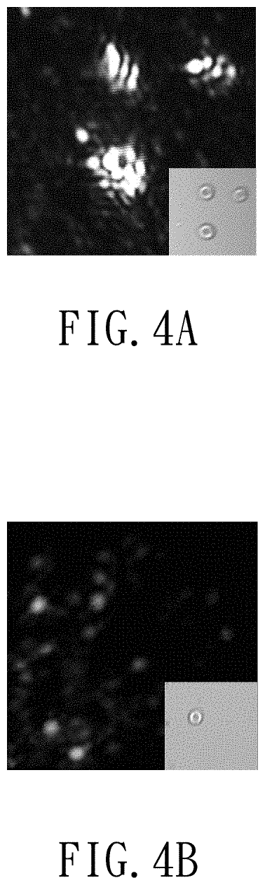

FIG. 2A is a schematic diagram of a detection result of a red blood cell observed by a scanning electron microscope. FIG. 2B is a schematic diagram of a detection result of a red blood cell and gold nanoparticle observed by a scanning electron microscope. FIG. 2C is a schematic diagram of a detection result of a A type red blood cell and gold nanoparticle bonding with anti-B antibody observed by a scanning electron microscope. FIG. 2D is a schematic diagram of a detection result of a A type red blood cell and gold nanoparticle bonding with anti-A antibody observed by a scanning electron microscope. Referring to FIG. 2A to FIG. 2D, a red blood cell used in the embodiment is A type red blood cell RC. When the gold nanoparticle G is not bonded to any antibody, there is almost no gold nanoparticle G attached to a surface of the red blood cell RC (as shown in FIG. 2B). When the gold nanoparticle G is bonded to anti-B antibody, as shown in FIG. 2C, it can be seen that a few gold nanoparticles G attach to the surface of the A type red blood cell RC as compared with FIG. 2B. When the gold nanoparticle G is bonded to anti-A antibody, as shown in FIG. 2D, it can be seen that a large amount of the gold nanoparticles G adhere to the surface of the A type red blood cell RC.

Taking A type antigen and anti-A antibody-golden nanoparticle as an example, since a single red blood cell has a plurality of A type antigens, when the antigen-antibody reaction occurs, a plurality of gold nanoparticles are attached to the surface of the single red blood cell via a combination of the A type antigen and the anti-A antibody. Although the gold nanoparticles do not cause aggregation with each other, at this time, a plurality of gold nanoparticles attached to the surface of the single red blood cell may be regarded as a whole, that is, a particle having a larger particle diameter. According to the scattering theory, the efficiency of scattering is characterized by its cross section σ_{scattering} and is proportional to the sixth power of a particle size R, and the formula is as follows:

$${σ}_{scattering} = \frac{128 {π}^{5}}{3 {λ}^{4}} {R}^{6} {\vert \frac{{m}^{2} - 1}{{m}^{2} - 1} \vert}^{2}$$

Therefore, it is expected that the intensity of the scattered light beam scattered by the plurality of gold nanoparticles attached to the surface of the single red blood cell (FIG. 2D) may be greater than the intensity of the scattered light beam scattered by the plurality of single gold nanoparticles suspended in the mixed solution.

Also, by designing the threshold of different requirements, it is possible to determine whether an antigen-antibody reaction occurs by detecting the intensity of the scattered light beam. The same applies to the other antigens and antibodies of the embodiment.

In the detection method of antigen-antibody reaction of the embodiment, there is a bond between the blood group specific antibody and the gold nanoparticle. When the blood group specific antibody reacts with the antigen on human red blood cell to form an antigen-antibody, since the gold nanoparticle itself may scatter the light beam and a large amount of the gold nanoparticle causes the scattered light beam to be greatly increased, by detecting the enhanced signal of the scattered light beam, it is possible to quickly determine whether an antigen-antibody reaction occurs, and there is no need to wait for the antigen-antibody reaction to be visible to the naked eyes, such as the phenomenon of red blood cells aggregation in the conventional agglutination reaction. In addition, the antigen detecting process of the invention does not require any washing step, so that the presence of the antigen may be detected in a minimum of 5 seconds, and the time required to detect the antigen-antibody reaction may be greatly reduced.

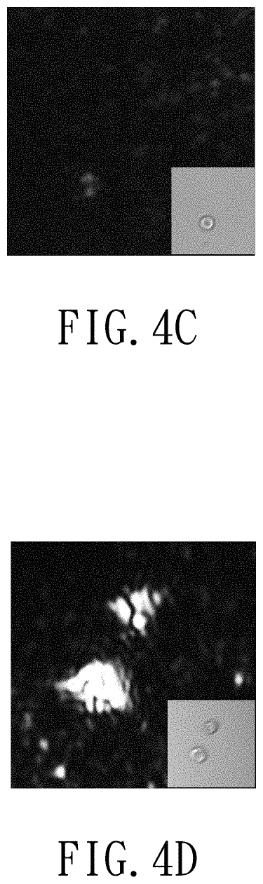

FIG. 3 is a schematic diagram of a detection device of one embodiment of the invention. Referring to FIG. 3, the method to detect the scattered light beam in the embodiment is, for example, using a detection device 10 including a light source 100 and an objective type dark field microscope 200. The light source 100 is, for example, a laser light source, but is not limited thereto. The light source 100 is adapted to provide a light beam L1. The objective type dark field microscope 200 includes a slide 210, a microscope objective 220, a mask 230 and a first light sensing element 240. The slide 210 is adapted to carry a mixed solution S. The mixed solution S here is an appropriate volume from the above mixed solution and added dropwise onto the slide 210. The microscope objective 220 is disposed on a side of the slide 210 away from the mixed solution S. The light beam L1 passes through the microscope objective 220 along a light incident direction A, then passes through the slide 210, and is irradiated to the mixed solution S, wherein partial of the light beam L1 is reflected by the slide 210 as a reflected light beam L2, and partial of the light beam L1 is scattered by the gold nanoparticles (not shown in FIG. 3) in the mixed solution S to form a scattered light beam L3. The reflected light beam L2 and the scattered light beam L3 are incident to the microscope objective 220, and are emitted in a light exiting direction B opposite to the light incident direction A. The mask 230 is disposed on a transmission path of the reflected light beam L2 and the scattered light beam L3, and is adapted to allow the scattered light beam L3 to pass and block a passage of the remaining light beams (such as the reflected light beam L2 of FIG. 3). The mask 230 has, for example, an opening 231 through which the light beam may pass, and the scattered light beam L3 passes through the mask 230 via the opening 231, but is not limited thereto. The first light sensing element 240 is disposed on a transmission path of the scattered light beam L3 and is adapted to receive the scattered light beam L3.

The detection device 10 may further include other optical elements, such as lenses 300, 310, 320, mirrors 400, 410 and aperture 500. The lenses 300, 310 and the mirror 400 are disposed between the light source 100 and the microscope objective 220. The lens 320 and the mirror 410 are disposed between the mask 230 and the first light sensing element 240. The aperture 500 is disposed between the mask 230 and the first light sensing element 240 and is adapted to further filter the reflected light beam L2 and allow the scattered light beam L3 to pass.

The first light sensing element 240 is, for example, a camera, but is not limited thereto. The camera may image the received scattered light beam L3 and determine whether an antigen-antibody reaction occurs by a brightness of the image.

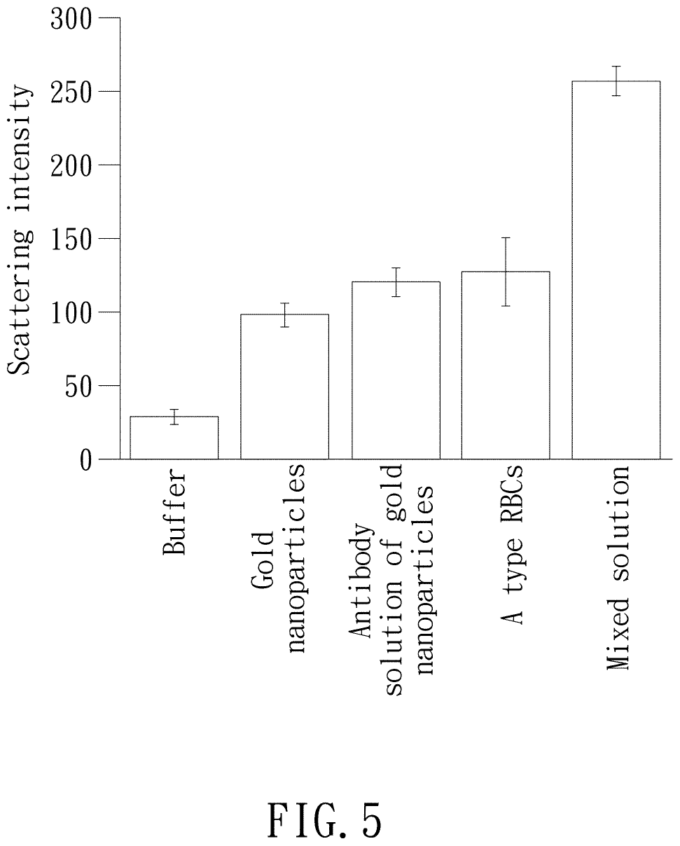

FIG. 4A to FIG. 4D are schematic diagrams of detection results of ABO blood group system using a detection device of FIG. 3. Referring to FIG. 4A to FIG. 4D, FIG. 4A shows a result of using red blood cells with A type antigen and anti-A antibody. FIG. 4B shows a result of using red blood cells with B type antigen and anti-A antibody. FIG. 4C shows a result of using red blood cells with A type antigen and anti-B antibody. FIG. 4D shows a result of using red blood cells with B type antigen and anti-B antibody. A small image in the lower left corner of each figure shows the results of red blood cells observed under a bright field microscope to cross-check whether the detection results are correct. As can be seen from the figures, positions of the red blood cells in FIGS. 4A and FIGS. 4D are the same as relative positions of bright spots, indicating that the antigen-antibody reaction is indeed detected. On the other hand, in FIGS. 4B and FIGS. 4C, since no significant antigen-antibody reaction occurs, no bright spots are observed.

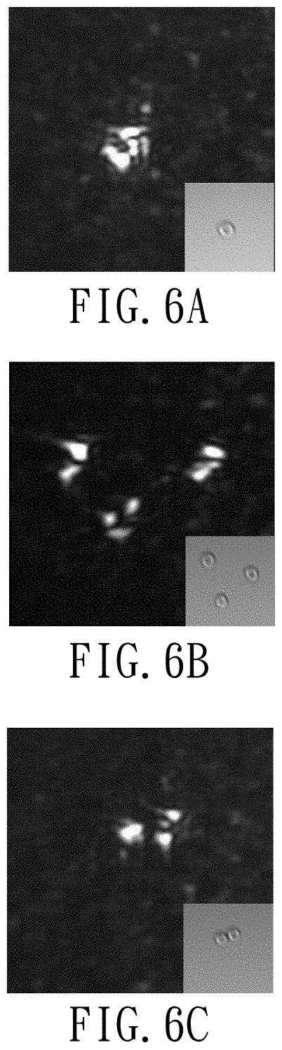

FIG. 5 is a schematic diagram of a scattering intensity result of FIG. 4. Referring to FIG. 3 to FIG. 5, the A type red blood cells and the anti-A antibody were used in the embodiment. FIG. 5 is sorted according to the magnitude of the scattering intensity, the scattering intensity values of the gold nanoparticles, the antibody solution of gold nanoparticles, and the A type red blood cells are similar, and the mixed solution has a higher scattering intensity value than the former.

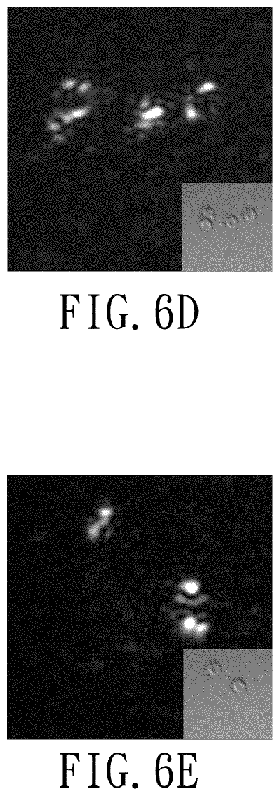

FIG. 6A to FIG. 6E are schematic diagrams of detection results of Rh blood group system using a detection device of FIG. 3. Referring to FIG. 6A to FIG. 6E, FIG. 6A shows a result of using red blood cells with D type antigen and anti-D antibody. FIG. 6B shows a result of using red blood cells with C type antigen and anti-C antibody. FIG. 6C shows a result of using red blood cells with E type antigen and anti-E antibody. FIG. 6D shows a result of using red blood cells with c type antigen and anti-c antibody. FIG. 6E shows a result of using red blood cells with e type antigen and anti-e antibody. FIG. 6A to FIG. 6E is presented in the same manner as FIG. 4A to FIG. 4D and will not be repeated herein.

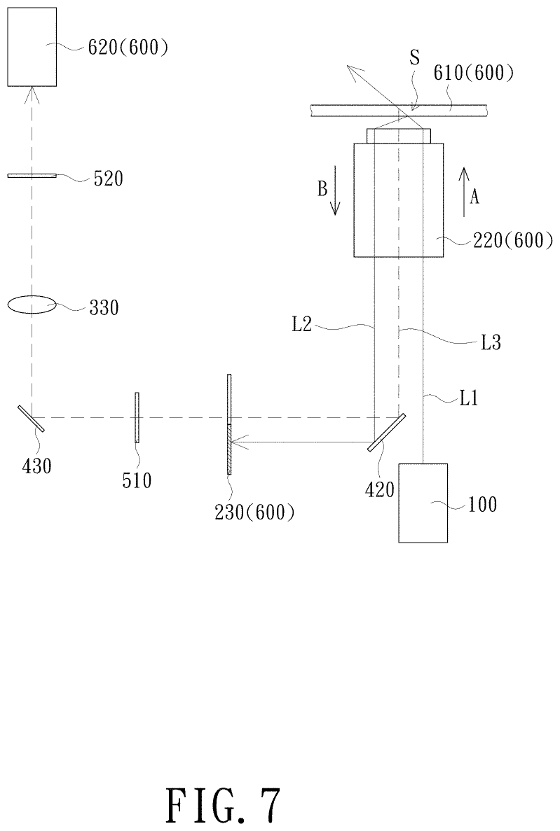

FIG. 7 is a schematic diagram of a detection device of another embodiment of the invention. Referring to FIG. 7, the detection device 10a of the embodiment is similar in structure and advantages to the detection device 10 described above, and only the main differences in the structure will be described below. The detecting device 10a of the embodiment includes the light source 100 and a capillary column detection device 600. The capillary column detection device 600 includes a capillary column 610, the microscope objective 220, the mask 230 and a second light sensing element 620. The mixed solution S is injected into the capillary column 610 by pressure and the solution can be changed at any time. The microscope objective 220 is disposed adjacent to the capillary column 610. The light beam L1 passes through the microscope objective 220 along a light incident direction A, and is incident to the capillary column 610 and irradiated to the mixed solution S, wherein partial of the light beam L1 is reflected by a side wall of the capillary column 610 as a reflected light beam L2, and partial of the light beam L1 is scattered by the gold nanoparticles (not shown in FIG. 3) in the mixed solution S to form a scattered light beam L3. The reflected light beam L2 and the scattered light beam L3 are, for example, incident to the microscope objective 220, and are emitted in a light exiting direction B opposite to the light incident direction A. The mask 230 is disposed on a transmission path of the reflected light beam L2 and the scattered light beam L3. The second light sensing element 620 is disposed on a transmission path of the scattered light beam L3 and is adapted to receive the scattered light beam L3.

The detecting device 10a may further include other optical elements such as a lens 330, mirrors 420, 430, and apertures 510, 520. The lens 330, the mirror 430, and the apertures 510, 520 are disposed between the mask 230 and the second light sensing element 620. The mirror 420 is disposed between the microscope objective 220 and the mask 230.

The second light sensing element 620 is, for example, a photomultiplier, but is not limited thereto. The photomultiplier is adapted to convert a light beam signal into an electrical signal and amplify it. After interpreting the value through the device, it is determined whether an antigen-antibody reaction occurs.

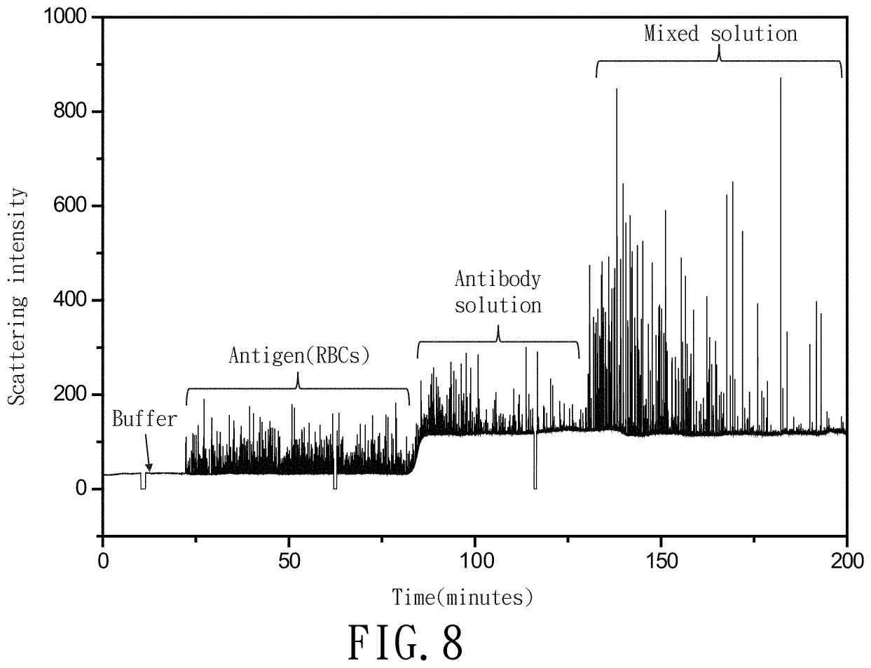

FIG. 8 is a schematic diagram of a detection result using the detection device of FIG. 7. Referring to FIG. 8, an unit of a vertical axis is scattering intensity, and an unit of a horizontal axis is time. The detection result uses a continuous injection method, which is divided into four stages from left to right in FIG. 8. The signal value of the first stage represents the injection of the buffer for 10 minutes. The signal value of the second stage represents the injection of antigen (red blood cells) for 50 minutes. The signal value of the third stage represents the injection of the antibody solution for 50 minutes. The signal value of the fourth stage represents the injection of the mixed solution for 50 minutes. The results show that the mixed solution of the fourth stage shows a higher signal average value than the previous three stages, thereby determining whether an antigen-antibody reaction occurs.

In summary, in the detection method of antigen-antibody reaction of the embodiment of the invention, there is a bond between the antibody and the type of metal nanoparticle. When the antibody reacts with the antigen to form an antigen-antibody, since the nanoparticles of the type of metal nanoparticle are close to each other, the scattered light beam can be greatly increased, by detecting the intensity signal of the scattered light beam, it is possible to quickly determine whether an antigen-antibody reaction occurs, and there is no need to wait for the antigen-antibody reaction to be visible to the naked eyes. In addition, the antigen detecting process of the invention does not require any washing step, so that the presence of the antigen may be detected in a minimum of 5 seconds, and the time required to detect the antigen-antibody reaction may be greatly reduced.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A detection method of antigen-antibody reaction, comprising:
providing an antibody solution, comprising an antibody and a type of metal nanoparticle, the type of metal nanoparticle forms bond with the antibody;
adding an antigen to the antibody solution to form a mixed solution;
providing a light beam to the mixed solution, wherein part of the light beam is scattered by the type of metal nanoparticle to form a scattered light beam; and
detecting the scattered light beam to determine whether an antigen-antibody reaction occurs.

2. The detection method of antigen-antibody reaction according to claim 1, wherein the type of metal nanoparticle comprises gold nanoparticle.

3. The detection method of antigen-antibody reaction according to claim 1, wherein the antigen comprises at least one of A type antigen, B type antigen, D antigen, C antigen, E antigen, c antigen, and e antigen on human red blood cells.

4. The detection method of antigen-antibody reaction according to claim 1, wherein the antibody comprises a specific antibody.

5. The detection method of antigen-antibody reaction according to claim 1, wherein the method to determine whether an antigen-antibody reaction occurs comprises: detecting whether an intensity of the scattered beam exceeds a threshold, and it is determined that an antigen-antibody reaction occurs if the intensity exceeds the threshold.

6. The detection method of antigen-antibody reaction according to claim 1, wherein the method to detect the scattered light beam comprises: using a detection device, comprising a light source and an objective type dark field microscope.

7. The detection method of antigen-antibody reaction according to claim 6, wherein the objective type dark field microscope comprises:
a slide, adapted to carry the mixed solution;
a microscope objective, disposed on a side of the slide away from the mixed solution, the light beam passes through the microscope objective and is irradiated to the mixed solution;
a mask, disposed on a transmission path of the scattered light beam and adapted to allow the scattered light beam to pass and block a passage of the remaining light beams; and
a first light sensing element, disposed on the transmission path of the scattered light beam and adapted to receive the scattered light beam

8. The detection method of antigen-antibody reaction according to claim 1, wherein the method to detect the scattered light beam comprises: using a detection device, comprising a light source and a capillary column detection device.

9. The detection method of antigen-antibody reaction according to claim 8, wherein the capillary column detection device comprises:
a capillary column, the mixed solution is placed in the capillary column;
a microscope objective, disposed adjacent to the capillary column, the light beam passes through the microscope objective and is irradiated to the mixed solution;
a mask, disposed on a transmission path of the scattered light beam and adapted to allow the scattered light beam to pass and block a passage of the remaining light beams; and
a second light sensing element, disposed on the transmission path of the scattered light beam and adapted to receive the scattered light beam.

10. The detection method of antigen-antibody reaction according to claim 9, wherein the second light sensing element comprises a photomultiplier.
